(19) United States
(12) Patent Application Publication
(10) Pub. No.: US 2021/0090460 A1
(43) Pub. Date: Mar. 25, 2021

(54) Information Processing System

(71) Applicants: Hiroshima University, Higashi-Hiroshima-shi (JP); Hitachi, Ltd., Tokyo (JP)

(72) Inventors: Tsukasa FUNANE, Tokyo (JP); Hirokazu ATSUMORI, Tokyo (JP); Akihiko KANDORI, Tokyo (JP); Akiko OBATA, Tokyo (JP); Ayako NISHIMURA, Tokyo (JP); Yuto KOMATSU, Tokyo (JP); Takashi NUMATA, Tokyo (JP); Seiji HAMA, Higashi-Hiroshima-shi (JP); Toshio TSUJI, Higashi-Hiroshima-shi (JP)

(21) Appl. No.: 16/935,778

(22) Filed: Jul. 22, 2020

(30) Foreign Application Priority Data
Sep. 24, 2019 (JP) 2019-173257

(51) Int. Cl. G09B 19/00; A61B 5/16; A61B 5/11; A61B 5/00

(52) U.S. Cl. CPC G09B 19/00; A61B 5/742; A61B 5/1126; A61B 5/16

(57) ABSTRACT

An information processing system visualizes brain states to optimize rehabilitation programs for rapid feedback of their effects to a patient and healthcare staff, to thereby promote the efficiency of rehabilitation. An embodiment of the system includes: a storage section that stores test-result brain-state relationship information associating results of activities of multiple examinees subjected to predetermined tests with brain states of the examinees; an input section that accepts a first test result as the result of the activity of a first examinee subjected to the predetermined tests; a control section that estimates the brain state from the first test result on the basis of the test-result brain-state relationship information; and an output section that outputs the estimated brain state.

### BACKGROUND OF THE INVENTION

### 1. Field of the Invention

The present invention relates to an information processing system for estimating the brain states of an examinee.

### 2. Description of the Related Art

In the field of rehabilitation (referred to as rehab hereunder where appropriate) involving patients with cerebrovascular disease such as brain infarction (or stroke), one of the challenges the patients face is how to boost their motivation. In order to achieve this, it is preferable to feed the effects of rehab back to the patients.

Another challenge is how to improve the efficiency of rehabilitation. This requires feeding the effects of rehabilitation back to healthcare workers such as doctors, nurses, occupational therapists, physical therapists, and speech therapists (generically referred to as healthcare staff hereunder) as needed so that the rehab effects will be reflected in rehabilitation programs.

As part of the background art in the field of the present technology, there have been reports of the results of some simplified tests (e.g., clinical assessment for attention (CAT)) being associated with the site of brain infarction. One such report is from Taro Murakami, Seiji Hama, Hidehisa Yamashita, Keiichi Onoda, Seiichiro Hibino, Hitoshi Sato, Shuji Ogawa, Shigeto Yamawaki, and Kaoru Kurisu, “Neuroanatomic pathway associated with attention deficits after stroke,” Brain Research 1544, 25-32 (2014).

### SUMMARY OF THE INVENTION

In feeding the effects of rehabilitation back to the patients and healthcare staff at earlier timing, the challenge is how to promote the efficiency of rehabilitation by visualizing brain states and optimizing the rehab programs accordingly.

In order to feed the rehab effects back to the patients and healthcare staff, details of the patient's brain states are desired to be grasped by simple tests. For example, it is desired to find out details of the brain states by simple tests without resorting to testing by magnetic resonance imaging (MRI) or computed tomography (CT). It is further desired to figure out the brain states in more detail than in the case of using solely the results of the simplified tests (e.g., clinical attention assessment (CAT)), for example.

It is therefore an object of the present invention to provide an information processing system capable of estimating the brain states of an examinee by use of simple tests.

In solving the above problem and according to one aspect of the present invention, there is provided an information processing system including: a storage section configured to store test result and brain state relationship information associating results of activities of multiple examinees subjected to predetermined tests with brain states of the examinees; an input section configured to accept a first test result as the result of the activity of a first examinee subjected to the predetermined tests; a control section configured to estimate the brain state from the first test result on a basis of the test-result brain-state relationship information; and an output section configured to output the estimated brain state.

According to the present invention, it is possible to provide an information processing system capable of estimating the brain states of an examinee even through simple testing. The foregoing and other objects, structures and advantages of the present invention will become evident from a reading of the following detailed description of a preferred embodiment.

### BRIEF DESCRIPTION OF THE DRAWINGS

FIG. 1 is a view depicting a typical configuration of an information processing system;

FIG. 2 is a view depicting a typical hardware configuration of the information processing system;

FIG. 3 is a view that lists information on relationship between tests results and brain states;

FIG. 4 is a flowchart depicting a process of outputting brain states;

FIG. 5 is a view depicting a typical three-dimensional probability map of brain lesion sites;

FIG. 6 is a view depicting a simplified flow of data at the time of creating a probability map of brain lesions and reserve and remaining functions;

FIG. 7 is a view depicting a combination map of brain lesions and reserve and remaining functions;

FIG. 8 is a view that lists examples of information included in a brain lesion database (DB), a brain function database (DB), and a rehabilitation database (DB);

FIG. 9 is a view depicting a simplified flow of data with a preferred embodiment;

FIG. 10 is a view depicting a typical configuration of an information processing system that includes a biological data acquiring section and a characteristic amount extracting section;

FIG. 11 is a view depicting a typical configuration of an information processing system that includes a test-result brain-state relationship information learning section;

FIG. 12 is a view depicting a characteristic amount extracting screen given at the time of presenting a brain image based on finger-tapping performance;

FIG. 13 is a view depicting an example of presenting brain images based on finger-tapping performance; and

FIG. 14 is a view depicting a simplified process flow for estimating a brain lesion site by calculating total travel distances at the time of left and right finger tapping.

### DESCRIPTION OF THE PREFERRED EMBODIMENTS

A preferred embodiment of the present invention is described below with reference to the accompanying drawings. Throughout the different drawings, the same reference numerals designate substantially the same constituent blocks and constituent elements.

### First Embodiment

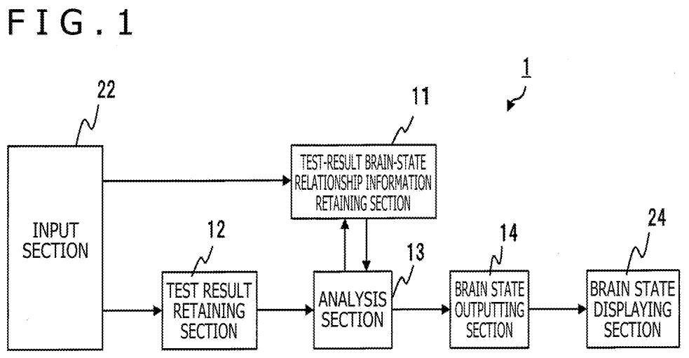

FIG. 1 depicts a typical configuration of an information processing system 1 embodying the present invention. The information processing system 1 is configured with a storage section, an input section, a control section, and an output section, for example. The information processing system 1 may further include a display section.

In the example of FIG. 1, the storage section includes a test result retaining section 12 and a test-result brain-state relationship information retaining section 11. The input section of the system corresponds to an input section 22, the control section to an analysis section 13, the output section to a brain state outputting section 14, and the display section to a brain state displaying section 24.

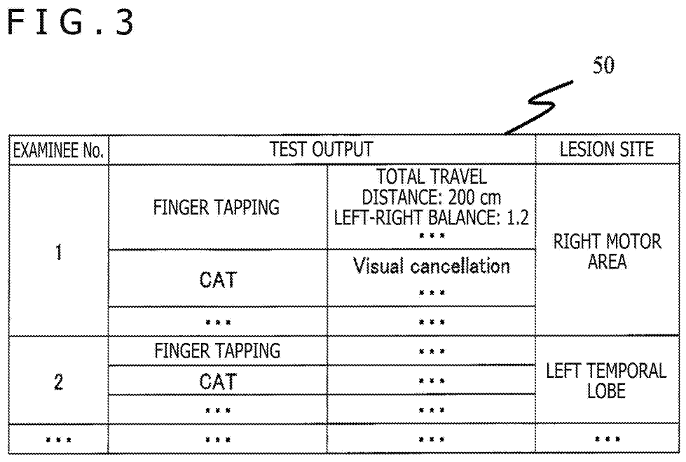

The storage section stores information on relationship between tests results and brain states, i.e., the information associating test results as the results of activities of examinees subjected to predetermined tests with brain states of the examinees. The information on relationship between tests results and brain states will be discussed later in detail with reference to FIG. 3. Here, examples of the predetermined tests may be tests of CAT or tests using biological measurements such as those with a finger tapping device. The examples of the predetermined tests may include tests on at least one of the brain functions regarding motion, cognition, and attention.

The input section accepts the results of activities of examinees subjected to the predetermined tests. A typical examinee is a patient. For example, a patient undergoing rehabilitation is expected to go through predetermined tests with a view to feeding the effects of rehab back to the patient and healthcare staff. Incidentally, the examinee may have the test results either included in the information on relationship between tests results and brain states or not included therein.

Given the test results from the input section, the control section estimates the brain states on the basis of the information on relationship between tests results and brain states. Here, the control section may estimate a brain lesion site as an example of the brain states.

The output section outputs the brain states estimated by the control section. Here, the output section may output an image indicative of the (estimated) brain lesion site. For example, the output section may visualize the brain states by outputting an image indicative of such states to a display device (display or monitor unit) acting as the display section, thereby feeding the effects of rehabilitation back to the patient and healthcare staff. The visualization makes it possible to have the feedback results reflected in rehabilitation programs (including as changes in the rehab programs) and to boost the patient's motivation. Further, rapid and effective offering and feedback of relevant information in such a manner to the patient and healthcare staff promotes the efficiency of rehabilitation.

In the case of cerebrovascular disease such as brain infarction (or stroke), a lesion site caused by infarction or stroke in the brain (including damage or defect site) can lead to a lost function. Further, the lost function varies depending on the lesion site in the brain. Thus, the results of activities of examinees subjected to predetermined tests indicate that a common brain lesion site in the examinees tends to come from the same or similar test results. On the other hand, different brain lesion sites in the examinees are highly likely to yield different test results. With this taken into account and given the results of the predetermined tests, this embodiment aims at estimating the brain states (e.g., brain lesion site) of the examinee on the basis of the information on relationship between test results and brain states. The predetermined tests may be simplified tests such as measurements by a finger tapping device. The information processing system 1 is capable of estimating the brain states of the examinee even by simplified tests.

Also, the embodiment improves inefficient rehabilitation involving mere repetition of rehab work on the function that has been lost due to the brain lesion site. Improved rehabilitation efficiency is expended to shorten the treatment period of rehabilitation. More efficient rehabilitation helps reduce the work load on the healthcare staff (healthcare workers such as doctors, occupational therapists, physical therapists and speech therapists) involved in rehabilitation. Furthermore, the improved rehab efficiency forestalls progress of the disease condition and prevents an oversight of a relapse.

In the example of FIG. 1, the test result retaining section 12 retains the test results obtained from multiple biological measurements such as those by clinical attention assessment (CAT) and by a finger tapping device. The analysis section 13 estimates a severe part in the probability map of brain lesion sites on the basis of the test results retained by the test result retaining section 12 and of the information on relationship between tests results and brain states retained by the test-result brain-state relationship information retaining section 11. The brain state outputting section 14 outputs three-dimensional data of a brain image from the estimated results to the brain state displaying section 24. The information on relationship between tests results and brain states may be retained in the form of a database by the test-result brain-state relationship information retaining section 11. Here, brain defect sites, brain infarction sites, and brain atrophy sites are generically referred to as the brain lesion site. The analysis section 13 estimates the brain states by inputting one or multiple parameters obtained from one or multiple test results retained by the test result retaining section 12 into the test-result brain-state relationship information retaining section 11 retaining the information on relationship between tests results and brain states.

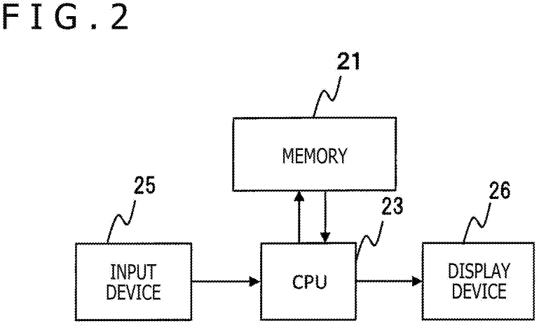

FIG. 2 is a view depicting a typical hardware configuration of the information processing system 1. The information processing system 1 is configured with a storage device, an input device 25, and an arithmetic device, for example. The storage device acts as a storage section, the input device 25 as an input section, and the arithmetic device as a control section and as an output section, for example. Preferably, the information processing system 1 may further include a display device 26 acting as a display section and a communication device acting as a communication section communicating with external devices. The arithmetic device may be configured using a processor such as a central processing unit (CPU) or a graphics processing unit (GPU) and may include dedicated circuits for performing specific processes. The dedicated circuits here include, for example, a field programmable gate array (FPGA), an application specific integrated circuit (ASIC), and complex programmable logic device (CPLD).

As explained below, this embodiment uses a memory 21 as the storage device and a CPU 23 as the arithmetic device, for example. The memory 21 constitutes the test result retaining section 12 and the test-result brain-state relationship information retaining section 11. The CPU 23 makes up the analysis section 13 and the brain state outputting section 14. The brain states output from the CPU 23 are displayed on the brain state displaying section 24 made of a display or monitor unit. In the embodiment, the CPU 23 acts as the control section and as the output section and, by performing programs retained in the memory 21, implements such functions as the analysis section 13 and the brain state outputting section 14. The analysis section 13, the brain state outputting section 14, a characteristic amount extracting section 62, and a test-result brain-state relationship information learning section 72, to be explained below, are implemented likewise by the CPU 23 of the embodiment to provide the respective functions.

The input device 25 acting as the input section 22 may be a mouse, a keyboard, and an interface that accepts data from external devices, for example. The input device 25 may alternatively be an input/output interface (IF). The display device 26 made of a display or monitor unit acts as the brain state displaying section 24. Preferably, the input/output IF and a communication channel may be provided interposingly between the brain state outputting section 14 and the brain state displaying section 24.

The information processing system 1 may be implemented in a hardware configuration that includes one or multiple computers (electronic computational machines). Incidentally, the above-described constituent elements of the hardware of the information processing system 1 may each be singular or plural in number.

FIG. 3 is a view that lists the content of a test vital-signal brain-lesion database 50 as an example of the information on relationship between tests results and brain states retained by the test-result brain-state relationship information retaining section 11. This is the information on relationship between tests results (results of tests or of biological measurements) and brain lesion sites. The information on relationship between tests results and brain states associates the test results as the results of activities of one or multiple examinees subjected to predetermined tests with the brain states of the examinees. In the example of the information on relationship between tests results and brain states in FIG. 3, identification information (examinee numbers) identifying the examinees (human examinees), test results (test output) as the results of activities of the examinees subjected to the predetermined tests, and the brain states (lesion sites) of the examinees are associated with one another. Here, examples of “the brain states of the examinees” in the information on relationship between tests results and brain states may be the brain states diagnosed by doctors using typically the results of brain imaging tests such as MRI and CT. Further examples of “the brain states of the examinees” may be brain images as well as information on brain structures and brain lesions obtained from the brain images. Furthermore, the examples of “the brain states of the examinees” may include information estimated regarding the brain states based on the brain images and various test results.

The test results, in the case of a finger-tapping test, provide such information as total travel distances, left-right balance, standard deviation of contact times, standard deviation of tapping intervals, and standard deviation of phase differences. In the case of CAT, the test results are those of digit span forward test, digit span backward test, visual cancellation test, and position stroop test, for example. The test results may further include scores of mini-mental state examination (MMSE) and functional independence measure (FIM). The test-result brain-state relationship information retaining section 11 retains such score information and characteristic amount information, as well as the information on brain structures and brain lesions from brain imaging tests such as MRI and CT. In this manner, the test-result brain-state relationship information retaining section 11 retains the information on relationship between diverse tests results and brain states.

The actual brain lesion site may not be limited to a single location and thus may not be represented by one region name. In this respect, the information on relationship between tests results and brain states may include information on brain infarction coordinates as well as information on the distribution of coordinate information. The test-result brain-state relationship information retaining section 11 may be configured to retain information on numerous examinees beforehand as a database for example. Thus configured, the test-result brain-state relationship information retaining section 11 may permit searches for the lesion site in an examinee corresponding to the score information from a given test. Incidentally, for this embodiment, the principal examinees are assumed to be humans. Thus, the examinee may also be referred to as the human examinee.

One specific method of calculating total travel distances may involve, for example, acquiring distances between the thumb and the index finger in chronological order and totaling twice the maximum amplitude of the distances in each test period to find the total travel distances of the right hand and left hand, before acquiring the total sum of these distances. That is, the distances are calculated from the physical positions of the fingers. The left-right balance may be calculated by finding the ratio of total travel distances between the left and right hands. The time of contact between the fingers may be obtained by defining both the time at which the thumb and the index finger are in contact with each other and the state in which the two fingers are apart from each other, and by adopting the time of the contact. The finger tapping interval may be calculated as the interval between the contact start time of the thumb and that of the index finger. The standard deviation of phase differences is calculated by performing, for example, a Hilbert transform on the chronological changes in left and right finger tapping so as to obtain phases therebetween and to calculate chronological changes of the difference in phase between the left and right hands. The standard deviation of the chronological changes of the difference in phase between the left and right hands may be acquired in this manner.

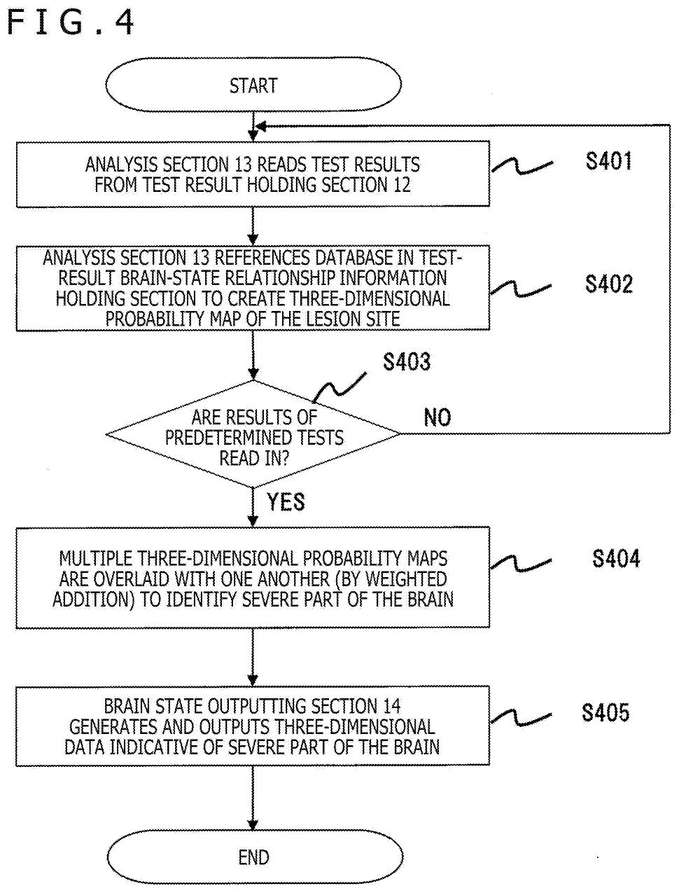

FIG. 4 is a flowchart depicting a process performed by the analysis section 13 for outputting a brain lesion site. This flowchart may be executed in a suitably timed manner, such as when the input section accepts an execution request from the administrator or from a management device, or when the input section 22 accepts test results representing the results of the activity of an examinee subjected to predetermined tests. First, the analysis section 13 reads the test results from the test result retaining section 12 (step S401).

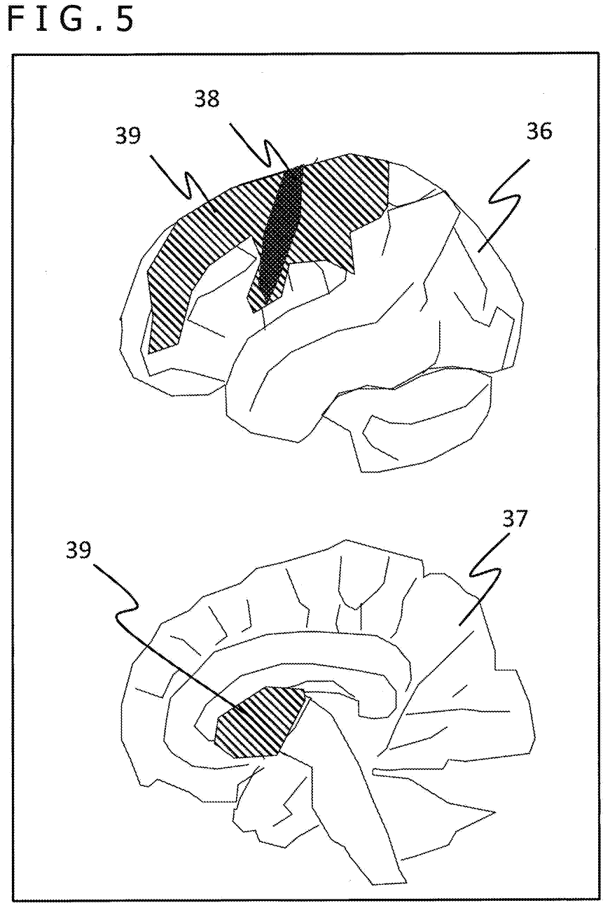

The analysis section 13 then references the information on relationship between tests results and brain states retained by the test-result brain-state relationship information retaining section 11 to create a three-dimensional probability map of brain lesion sites (step S402). FIG. 5 depicts a typical three-dimensional probability map of brain lesion sites. Whereas this embodiment uses a three-dimensional probability map as an example for explanation, any other type of map is acceptable as long as the map reveals brain states. On a three-dimensional brain model (on its surface) 36, a high-probability region 38 highly probable to be a brain lesion site and a low-probability region 39 are presented by different methods (e.g., by different hatch patterns, color differences, or shade differences). Although what is indicated here is a three-dimensional distribution of two probabilities, continuous probability values may be mapped using different shades in color or different colors. The mapping is not limited to the surface of the brain model. Alternatively, the regions may be mapped three-dimensionally in a three-dimensional brain model (inside) 37.

At the time of creating a three-dimensional probability map, the brain lesion sites corresponding to a given score are referenced from within a database (of the information on relationship between tests results and brain states) so as to map the actual brain lesion site of each examinee (by inverse projection). This process is carried out on all examinees included in the database. Part or all of the brain lesion sites of the examinees corresponding to the given score may be overlaid with one another and mapped to a reference brain (e.g., to the Montreal Neurological Institute (MNI) coordinate system) to calculate frequency information at the time of the mapping. The frequency information may then be mapped to the three-dimensional brain model (reference brain). The information mapped to the reference brain may be further mapped to brain images previously acquired of each examinee by MRI or CT. The mapping makes it possible to display a three-dimensional probability map corresponding to the frequency information on the brain lesion sites so as to present, for example, a brain lesion site that is highly likely common to multiple examinees corresponding to the given score (i.e., brain lesion site observed with high probability) as the high-probability region 38 in the three-dimensional brain model. Here, the examinees corresponding to the given score are not limited to the examinees of the same score. The examinees may be those belonging to a given range of scores (predetermined range) or those who manifest similar characteristics in scores. The examinees corresponding to the scores complying with such predetermined criteria may then be selected.

It is expected that the larger the number of examinees included in the database, the higher the accuracy of the probability map. Further, displaying the probability map on the brain image of each examinee enables more clear-cut feedback for each examinee. Here, the brain image of each examinee may be obtained by transforming a reference brain structure in keeping with brain structure information on, and the coordinate system of, each examinee.

Next, the analysis section 13 determines whether predetermined test results have been read (step S403). If the result of the determination in step S403 is negative (NO), step S401 is reached. In this manner, where the target examinee has multiple test results such as the test result of finger-tapping performance and the test result of CAT, a three-dimensional probability map is created for each of the tests.

If the result of the determination in step S403 is positive (YES), the three-dimensional probability maps of multiple lesion sites are overlaid with one another by weighted addition to identify a severe part of the brain (step S404). The method of display may be that as depicted in FIG. 5 (an example of three-dimensional probability map display), with the severe part highlighted on display. The severe part of the brain displayed here is a high-probability brain lesion site calculated using one or multiple lesion probability maps corresponding to various test results.

The brain state outputting section 14 then generates three-dimensional data indicative of the severe part of the brain and outputs the generated data (step S405). Alternatively, the brain state outputting section 14 may output the three-dimensional probability map in step S402. In this flowchart, steps S403, S404 and S405 may or may not be carried out. That is, some of the steps constituting the flowchart need not be performed, and additional steps may be carried out in the flowchart.

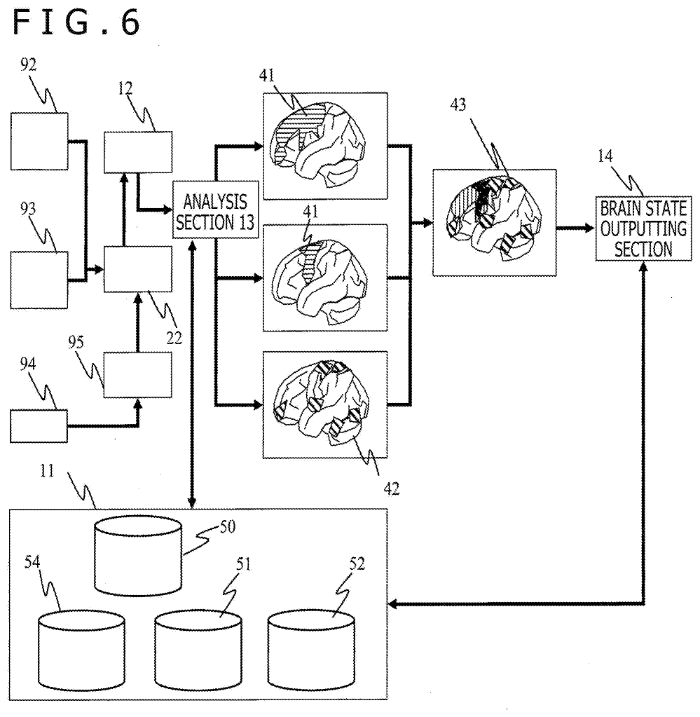

FIG. 6 is a view depicting a simplified flow of data at the time of creating a probability map of brain lesions and reserve and remaining functions. The input section 22 accepts the result of biological measurement 92, the result of tests 93, a score 95 as the result of an intervention 94, and intervention information. The input section 22 records what is accepted to the test result retaining section 12. Here, the biological measurement 92 stands for finger tapping and the tests 93 denote CAT, for example. The score 95 represents information such as the level of intervention in the case where medical intervention or medical practice is carried out.

The information on relationship between test results and brain states may include, for example, at least part or all of the result of the biological measurement 92 (e.g., vital signals), the result of the tests 93, the score 95 as the result of the intervention 94, and the intervention information associated with the brain states of one or multiple examinees.

Given input of the result of the biological measurement 92, the result of the tests 93, the score 95, or the intervention level information with respect to the target examinee, the analysis section 13 searches the database (DB) retained by the test-result brain-state relationship information retaining section 11 in order to estimate a brain lesion map 41 derived from the respective biological information (test results).

A typical brain lesion map 41 is the three-dimensional probability map of brain lesion sites as depicted in FIG. 5. The method of creating the brain lesion map 41 may be similar to the method explained above with reference to FIG. 4. For example, one or multiple examinees may be selected who have the test results corresponding to (identical to, equivalent to, or having similar tendencies toward) the test results of the target examinee (e.g., result of the biological measurement 92, result of the tests 93, score 95, and intervention information) in the information on relationship between tests results and brain states. The brain lesion sites of the selected one or multiple examinees may then be overlaid with one another on the reference brain (e.g., MNI coordinate system) before being mapped thereto to create the brain lesion map 41.

A brain activity map 42 may be created as follows: For example, of the brain lesion sites of the target examinee, those sites expected, based on the result of the biological measurement 92 associated with the brain function (left hand action or the like) or intervention information, to be active or become active when affected by intervention may be estimated from a brain-site brain-function database 51 or from a intervention brain-site database 54. The estimated brain sites may then be mapped to the brain model to create the brain activity map 42.

The brain activity map 42 need not be created by actually measuring brain activities in brain imaging assessment. Instead, the brain activity map 42 may be created by estimation using databases acquired from medical literature and biological measurements. In the case where the brain sites estimated to be active are displayed concurrently with the estimated brain lesion on the brain activity map 42, the brain sites may be referred to as the reserve and remaining function sites where appropriate in this description.

The databases stored in the test-result brain-state relationship information retaining section 11 include probability maps of brain lesions corresponding to clinical tests and vital signal levels (test vital-signal brain-lesion database 50), the brain-site brain-function database 51 that retains the active sites associated with the brain functions related to biological information, the intervention brain-site database 54 that retains the brain sites expected to become active when affected by intervention, and a brain-function rehabilitation database 52.

Using the above items of information, the analysis section 13 creates multiple brain lesion maps 41 and the brain activity map 42. By combining these maps, the analysis section 13 further creates a combination map 43 that indicates the sites of brain lesions and of reserve and remaining functions. The brain state outputting section 14 outputs part or all of the brain lesion map 41, brain activity map 42, and combination map 43 to the brain state displaying section 24, for example. In the description that follows, the maps indicative of brain states such as the brain lesion map 41, brain activity map 42, and combination map 43 may be referred to as brain maps 34. The brain states output from the brain state outputting section 14 may include information about the sites of brain lesions and the reserve and remaining functions estimated by the analysis section 13. This provides an information processing system that permits simple grasping of the brain states through visualization of the brain lesion sites and of the reserve and remaining function sites.

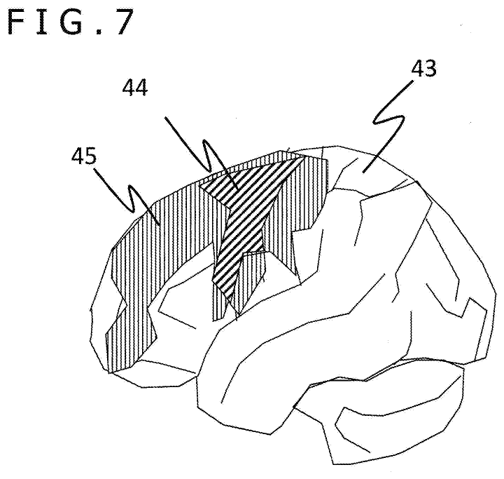

FIG. 7 depicts a typical combination map 43 indicative of the sites of brain lesions and reserve and remaining functions. For example, of the regions that are estimated to have a lesion, the one in which brain activity is expected on the basis of vital signals or intervention scores is displayed as a reserve and remaining function site 45. The other regions are indicated as a lesion site 44.

Displaying the reserve and remaining function sites in this manner provides an advantageous effect of knowing the possibility of an alternative site replacing the function that ought to be assumed by the lesion site. Also, carrying out the above estimation with time permits display, over time, of the lesion sites and the sites of reserve and remaining functions. This makes it possible to visualize the sites in which changes have been brought about by rehabilitation or by treatment, thereby providing instantaneous feedback of the effect of such rehab or treatment to the patient and healthcare staff. That in turn provides an advantageous effect of optimizing the rehabilitation program in use.

In addition to outputting the combination map 43 indicative of brain lesions and the sites of reserve and remaining functions, the brain state outputting section 14 may output information on the brain functions associated with the lesion site and a recommended rehabilitation plan in the form of a report.

Using the brain-site brain-function database 51, the brain state outputting section 14 outputs information regarding, for example, left hand action, language function, pain, repression function, and attention function as the information on the brain functions associated with the lesion site. Using the brain-function rehabilitation database 52, the brain state outputting section 14 outputs information such as a daily plan, a monthly plan, a self-training plan, and a recommended treatment as recommended rehabilitation plans for training predetermined brain functions (e.g., functions assumed by the patient's reserve and remaining function sites). In this case, the brain state outputting section 14 may output information on the frequency of making actual rehabilitation records included in the brain-function rehabilitation database 52.

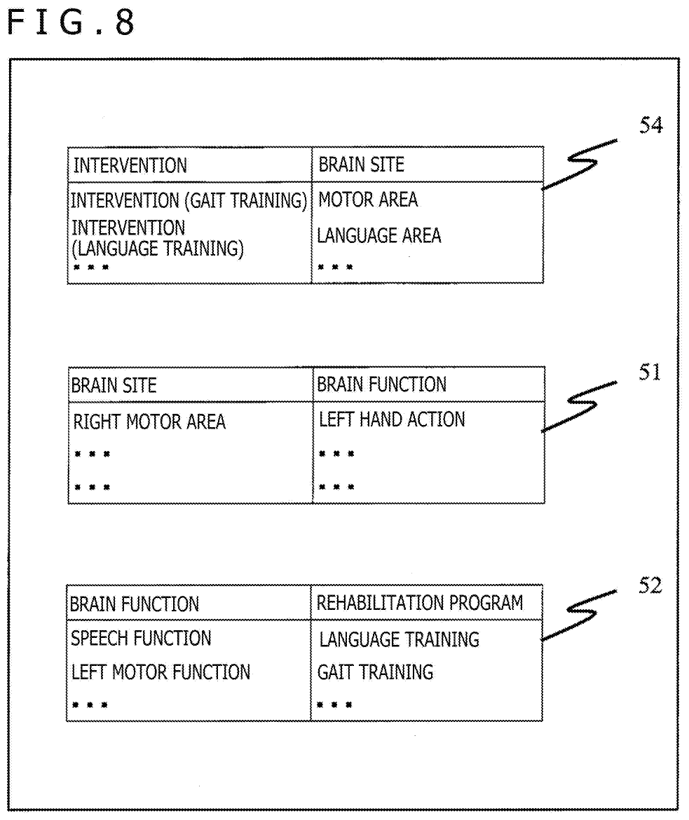

FIG. 8 lists examples of information included in the intervention brain-site database 54 (also referred to as the brain site database hereunder), in the brain-site brain-function database 51 (referred to as the brain function database), and in the brain-function rehabilitation database 52 (referred to as the rehabilitation database).

The brain site database 54 includes, for example, information that associates intervention information with the brain sites affected by interventions. The typical brain site database 54 in FIG. 8 retains information on the motor area as the region expected to be activated by an intervention (gait training), the language area as the region expected to be activated by another intervention (language training), and so on. The brain site database 54 and the brain function database 51 are created using diverse literature information and biological measurements.

The brain function database 51 retains information regarding the types of brain functions corresponding to predetermined brain sites. A typical brain function database 51 in FIG. 8 retains, for example, information on the right motor area associated with the left hand action as the corresponding function. The test-result brain-state relationship information retaining section 11 acting as the storage section retains the brain function database 51 that associates brain sites with brain functions.

The rehabilitation database 52 retains examples of specific rehabilitation programs (e.g., language training and gait training) for training the specific corresponding brain functions (e.g., speech function and left motor function). The rehabilitation database 52 is created on the basis of past experiences, literature, and preceding databases. Retaining the above databases insubstantial quantities or having them ready for use makes it possible to create, with higher accuracy, the combination map 43 indicative of brain lesions and the sites of reserve and remaining functions as depicted in FIG. 7. Creating such a highly accurate combination map enables the proposal of appropriate rehabilitation programs. The test-result brain-state relationship information retaining section 11 acting as the storage section retains the rehabilitation database 52 that associates the brain functions with the rehab programs for training these brain functions.

The above process makes it easy to know the brain states without measuring the brain and thereby to visualize the effects of rehabilitation and treatment by medication or the like. The visualization provides advantageous effects of leading to improvement of rehabilitation plan or treatment plan and to increasing the motivation of the patient and his/her family.

Further, retaining the database including the relationships between the effects of rehabilitation or of treatment by medication or the like and the rehab programs, the rehabilitation program optimized for the brain state can be created. This provides an advantageous effect of alleviating the workload on the healthcare staff such as doctors, occupational therapists, physical therapists, and speech therapists involved in rehabilitation work.

Furthermore, the brain states are visualized at regular intervals subsequent to rehabilitation or treatment by medication or the like. This provides an advantageous effect of preventing an oversight of progress or a relapse.

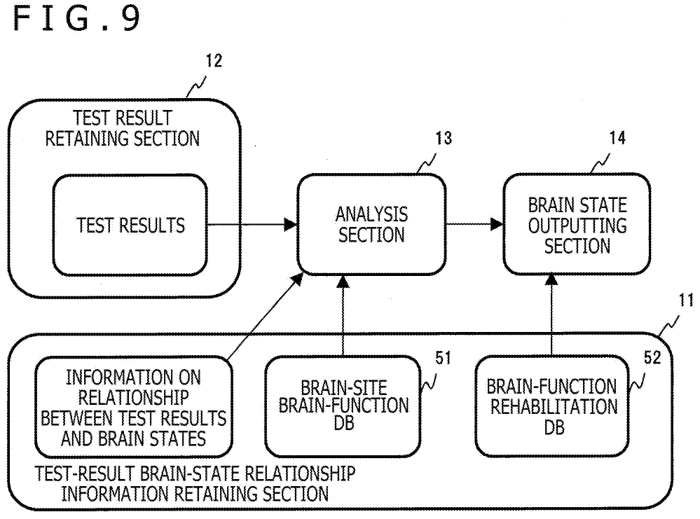

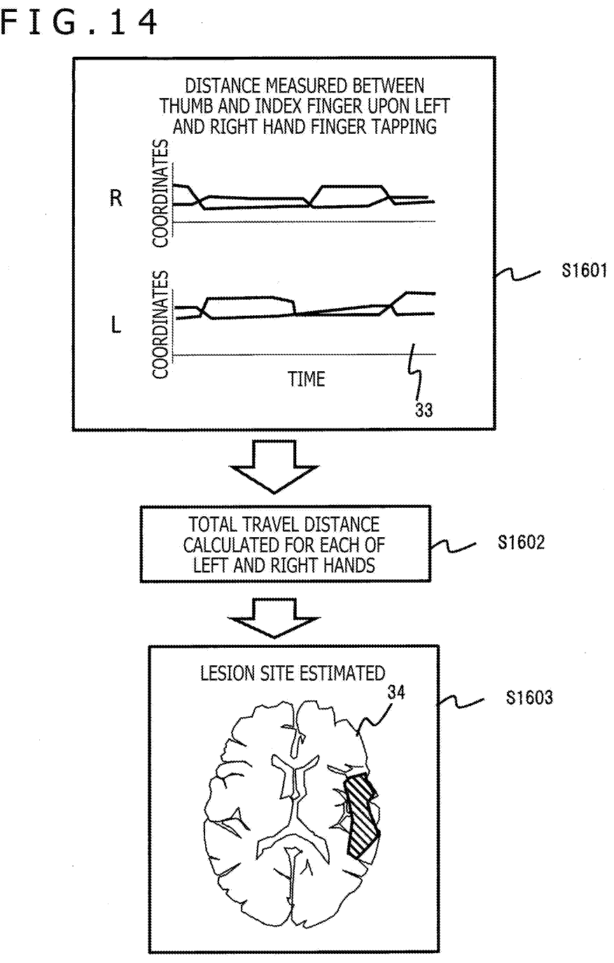

Next, FIG. 9 depicts a simplified flow of data with the embodiment. The analysis section 13 compares the results of tests such as CAT held by the test result retaining section 12 with the information on relationship between tests results and brain states retained by the test-result brain-state relationship information retaining section 11, so as to estimate a brain lesion site (e.g., defect site or reserve and remaining function site of the brain) using the inference method of pattern matching or of machine learning, for example, thereby creating a brain map 34 (see FIG. 14). As one example of the brain map 34, the brain state outputting section 14 outputs three-dimensional coordinate data of the brain indicative of a brain lesion site based on inverse projection mapping of the brain, and performs control to display the map on the brain state displaying section 24.

With the defect site or the reserve and remaining function site of the brain thus grasped, the analysis section 13 references the brain-site brain-function database 51 depicted in FIG. 8 to identify the brain function associated with the above estimated brain lesion site (e.g., defect site or reserve and remaining function site of the brain). With the brain function thus identified, the brain state outputting section 14 references the rehabilitation database 52 to output the rehabilitation program associated with the identified brain function.

The brain state outputting section 14 may cause the brain state displaying section 24 to display, either simultaneously or separately, the defect site or the reserve and remaining function site of the brain estimated by the analysis section 13 with simplified tests and the information on the identified rehabilitation program.

The analysis section 13 estimates the brain site or the brain lesion site in which the brain function activity is reduced, according to the test results of the examinee on the basis of the information on relationship between tests results and brain states and of the brain function database 51 retained by the test-result brain-state relationship information retaining section 11. Further, the brain state outputting section 14 performs control to display, on the brain model, the estimated brain site or brain lesion site in which the brain function activity is reduced.

The input section 22 inputs to the rehabilitation database 52 the results of rehabilitation as indicators such as the functional independence measure (FIM) indicative of the activity of daily living (ADL), thereby updating the database.

The brain state outputting section 14 may cause the brain state displaying section 24 to display the brain lesion site estimated by simplified tests and the rehabilitation program. The examinee is prompted to undergo the rehabilitation program periodically and to verify changes over time in the brain map using the information processing system 1. In this manner, the examinee can confirm the effects of the rehabilitation program.

With regard to the target patient, the analysis section 13 records to the test result retaining section 12 the results of the tests including the time at which the examinee was tested in association with the brain lesion site estimated from the test results. The test result retaining section 12 may retain, regarding the target examinee, not only the brain lesion site estimated from the most recent tests but also any brain lesion site that was estimated from previous tests.

Given the results of predetermined tests carried out at different times on the target examinee, the analysis section 13 estimates the respective brain states at the different times on the basis of the information on relationship between tests results and brain states. The brain state outputting section 14 outputs the estimated brain states or changes over time in the brain state. Here, the predetermined tests carried out at different times may be the test performed before a given rehabilitation program and the test carried out thereafter. The tests thus conducted make it possible to visualize the changes over time in the brain state before and after the rehabilitation program of interest using the display on (i.e., output to) the brain state displaying section 24. The visualization allows the patient and healthcare staff to confirm the effects of the rehab program. The information on the rehabilitation program carried out at different times as mentioned above may be output together with the estimated respective brain states at the different times or the changes over time in the brain state.

The brain state outputting section 14 also has a function of outputting the predetermined tests (such as cognitive tests) necessary for estimating the brain states. For example, the test-result brain-state relationship information retaining section 11 may be arranged to store beforehand the information on the tests needed to estimate the brain states. By referencing this information, the brain state outputting section 14 may output to the brain state displaying section 24 the predetermined tests such as CAT necessary for confirming the effects of rehabilitation. The brain function database 51 may be a database made available at a website or offered in the form of a table that lists relationship between coordinates and keywords.

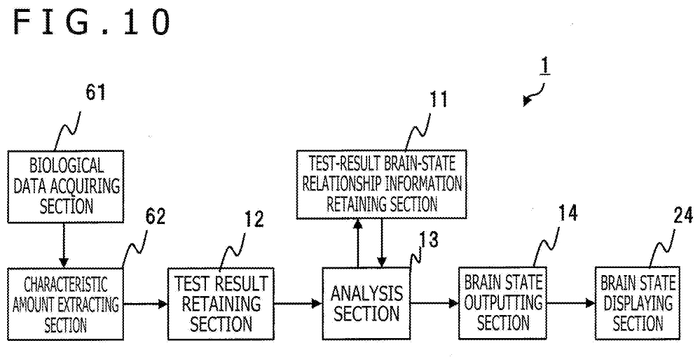

FIG. 10 depicts the information processing system 1 that additionally includes a biological data acquiring section 61 and a characteristic amount extracting section 62. The characteristic amount extracting section 62 is constituted by the CPU 23 and the biological data acquiring section 61 by the input section 22.

The biological data acquiring section 61 acquires biological data. The characteristic amount extracting section 62 extracts characteristic amounts from the biological data. The test result retaining section 12 retains the biological data and the characteristic amounts as the test results. The characteristic amounts here include, for example, travel distance, finger movement energy, finger contact time, finger tapping interval, and finger tapping phase in the case where the finger tapping device is used; or finger tapping speed, variations in finger tapping timing, and variations in finger tapping distance in the case where finger tapping images are used.

Using the test results, the analysis section 13 estimates the brain states such as the brain lesion site from the information on relationship between tests results and brain states retained by the test-result brain-state relationship information retaining section 11. The brain state outputting section 14 outputs the result of the estimation. The brain state displaying section 24 displays the estimated brain states.

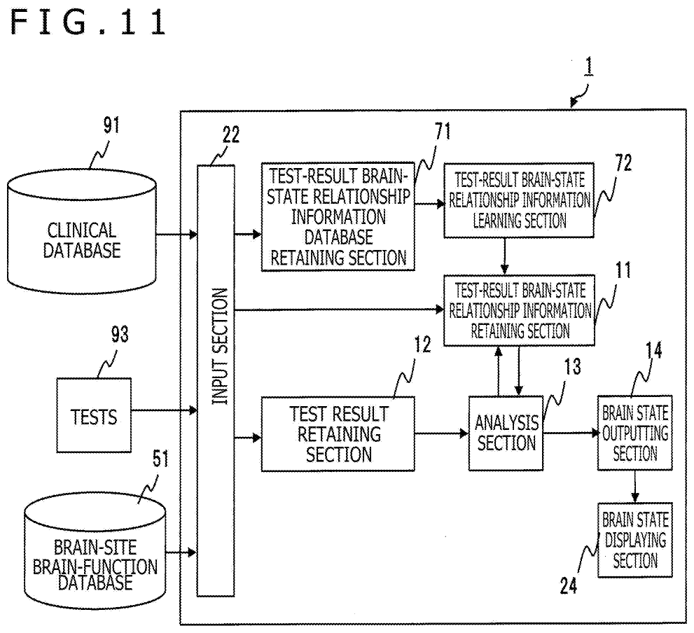

FIG. 11 depicts the information processing system 1 that includes a test-result brain-state relationship information learning section 72. The information processing system 1 illustrated in FIG. 1 further includes a test-result brain-state relationship information database retaining section 71 that retains the database of the information on relationship between test results and brain states, as well as the test-result brain-state relationship information learning section 72. An example of the information on relationship between test results and brain states may be information substantially the same as or equivalent to the test-result brain-state relationship information listed in FIG. 3. The information may further include brain images.

The control section of the information processing system 1 includes the test-result brain-state relationship information learning section 72. The test-result brain-state relationship information learning section 72 learns the relationship between test results and brain states using the database including the relationship between test results and brain states and retained by the test-result brain-state relationship information database retaining section 71. The test-result brain-state relationship information retaining section 11 retains the results of learning by the test-result brain-state relationship information learning section 72. The test-result brain-state relationship information database retaining section 71 is constituted by the memory 21 and the test-result brain state-relationship information learning section 72 by the CPU 23.

The test-result brain-state relationship information learning section 72 learns the relationship between test results (e.g., scores) and brain lesion sites using the database including the relationship between test results and brain states and retained by the test-result brain-state relationship information database retaining section 71. By so doing, the test-result brain-state relationship information learning section 72 can extract characteristics of the test results (e.g., score tendencies) common to multiple examinees having the same lesion site. The test-result brain-state relationship information learning section 72 records the characteristics as the learning results to the test-result brain-state relationship information retaining section 11. In estimating the brain states from the test results of a given examinee, the analysis section 13 determines whether the examinee has the characteristics that are substantially the same as the learning results. If the examinee has substantially the same characteristics, it is possible to estimate that the examinee has the same brain lesion site. Carrying out this type of learning is expected to boost the accuracy of estimating the brain states. Furthermore, learning in advance and performing estimates based on the learning results can shorten the time required to estimate the brain states.

The input section 22 accepts input from a clinical database 91, results of tests 93, and information from the brain function database 51. The clinical database 91 and the brain function database 51 may be stored in an external storage section, for example.

The clinical database 91 includes information on lesion sites such as the results of CAT tests, questionnaire-based tests, simplified motion measurement tests including finger tapping test, structured MRI tests, and CT tests. These items of information are stored as the information on relationship between test results and brain states (test-result brain-state relationship information) in the test-result brain-state relationship information database retaining section 71. The test-result brain-state relationship information database retaining section 71 further retains brain images input from the input section 22.

The results of the tests 93 are recorded to the test result retaining section 12.

The brain function database 51 includes information on corresponding relations between various brain sites and brain coordinates on one hand and the functions corresponding thereto on the other hand. This information is recorded to the test-result brain-state relationship information retaining section 11 as the information on relationship between brain sites and brain functions.

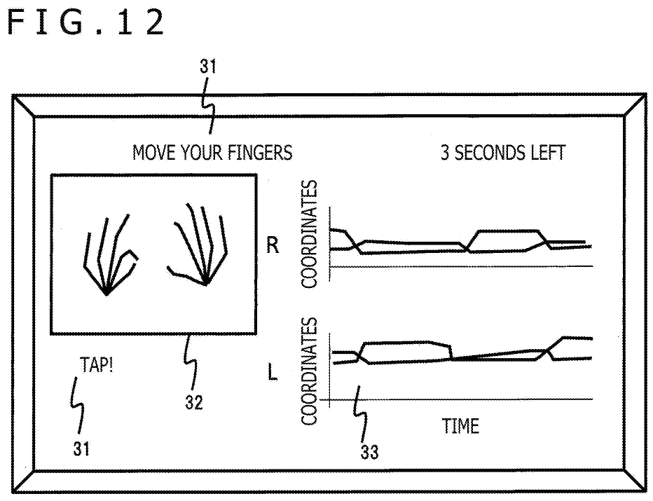

FIG. 12 is a view depicting a typical characteristic amount extracting screen given at the time of presenting a brain image based on finger-tapping performance. The brain state outputting section 14 causes the brain state displaying section 24 to display a screen that includes a test displaying section 31 indicative of information on test instructions. In the example of FIG. 12, instructions are given to perform finger tapping with both hands. In the information processing system 1, the input section 22 accepts images captured of the finger tapping (captured-image information) by a camera connected with the system 1, and the captured-image information is recorded to the test result retaining section 12. The brain state outputting section 14 causes the brain state displaying section 24 to display a screen including a captured-image information displaying section 32 indicative of the captured-image information. The display provides the examinee with feedback of whether the examinee is performing finger tapping correctly.

The test result retaining section 12 further includes records of the characteristic amounts from previous tests. Using the recoded characteristic amounts, the brain state outputting section 14 causes the brain state displaying section 24 to display a screen including a characteristic amount displaying section 33 indicative of the characteristic amounts over time. Here, the coordinates of the tips of the index finger and of the thumb are displayed chronologically for each of the right hand (R) and left hand (L).

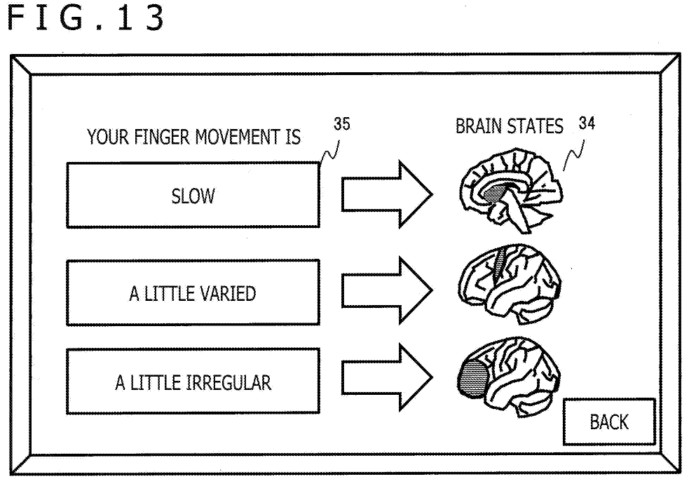

FIG. 13 is a view depicting an example of presenting brain images based on finger-tapping performance. The analysis section 13 determines the magnitude of each of the characteristic amounts such as the speed of finger tapping, the balance in magnitude between the right and left hands, and variations in phase between the right and left hands. By use of the results of the determination, the analysis section 13 estimates the presence or absence of a brain lesion. The brain state outputting section 14 causes the brain state displaying section 24 to display a screen including a characteristic amount determination result displaying section 35 indicative of the result of the determination on each of the characteristic amounts. If the results of the determination indicate that the scores on the characteristic amounts are worse than predetermined criteria, the brain state outputting section 14 displays, on the estimated brain maps 34, the probabilities of the corresponding brain sites having a brain lesion. In this case, the brain state outputting section 14 makes use of the information such as that in the test vital-signal brain-lesion database 50 retained by the test-result brain-state relationship information retaining section 11. Whereas the example here involves displaying the brain lesion site, what is displayed is not limited to the brain lesion site. Alternatively, the sites of reserve and remaining functions may be displayed, or a combination map 43 may be displayed to indicate the brain lesions and the reserve and remaining function sites in combination.

The analysis section 13 creates the brain maps 34 by calculating lesion probabilities into indicators and by mapping the calculated indicators. Where there are multiple tests that are predetermined, the analysis section 13 may estimate the lesion probabilities from these tests and have the estimated lesion probabilities overlaid with one another to create the brain maps 34. Preferably, the test-result brain-state relationship information learning section 72 may be arranged to learn all test patterns beforehand so as to have the learning results reflected in the test-result brain-state relationship information. Combining the results of multiple tests in this manner makes it possible to create maps more accurately. The test vital-signal brain-lesion database 50 includes brain structures, blood components, actual brain images, brain lesion sites, histories of rehabilitation programs, and information on the staff involved in rehabilitation work with respect to action measurement and analysis indicators (i.e., characteristic amounts). Using the information included in the database permits analysis of how these factors affect the action indicators, for example.

FIG. 14 depicts a simplified process flow for estimating a brain lesion site by calculating total travel distances at the time of left and right finger tapping. As indicated in the characteristic amount displaying section 33, the biological data acquiring section 61 first accepts images captured of the finger tapping (captured-image information) by a camera connected with the system 1. Given the captured images, the characteristic amount extracting section 62 calculates the distance between the thumb and the index finger at the time of left and right hand finger tapping (step S1601). The analysis section 13 then calculates the total travel distance for each of the right and left hands (step S1602). The analysis section 13 determines the magnitude of the total travel distance for each of the right and left hands as the characteristic amounts in accordance with predetermined criteria, and estimates the presence or absence of a brain lesion based on the results of the determination. For example, if the total travel distance of the left hand is extremely small, a lost function of the right motor area is suspected. Such estimates are derived from the test vital-signal brain-lesion database 50 retained by the test-result brain-state relationship information retaining section 11 (step S1603).

Although this embodiment has been described using simple examples, the information processing system 1, when faced with cases where multiple brain sites are involved, can also estimate brain lesion sites by inverse projection mapping of the brain through database search and output the estimated brain lesion sites in the form of a probability map.

According to the present embodiment, the test-result brain-state relationship information retaining section 11 may retain a database on the relationship between simplified tests (e.g., cognitive tests that can be performed using paper or a tablet) on one hand and the brain states such as lesion and infarction sites on the other hand. The database provides conditions conducive to estimating the brain disease state from the simplified tests through machine learning (inverse projection from the result to the cause), for example.

It is also possible to display, from simplified tests, brain abnormalities and the effects of intervention (rehabilitation) at the same time.

The analysis section 13 estimates the brain lesion site from multiple parameters of one or multiple simplified tests. The analysis section 13 can estimate the brain lesion site by having multiple probability maps overlaid with one another.

The analysis section 13, given an estimated lesion, may obtain a “suspected lost function” from databases (literature databases such as Neurosynth). The analysis section 13 may also obtain “expected remaining functions” assumed by the sites other than the lesion site from the databases described above. The information processing system 1 provides simplified tests on the function of interest (suspected lost function). In accordance with the results of these tests, the analysis section 13 estimates “remaining functions.” Where tests are carried out before and after rehabilitation, the events performed between the tests (e.g., rehab programs) may be retained in the form of a database by the test result retaining section 12. The analysis section 13 and the test-result brain-state relationship information learning section 72 may evaluate the relationship between the amounts of change in the test results on one hand and the rehabilitation programs on the other hand (e.g., by correlation analysis, principal component analysis, and machine learning). In this manner, the correspondence between specific rehabilitation programs and specific brain function improvements associated therewith may be visualized. Likewise, the duration of rehabilitation that patients have participated in and the duty hours of healthcare workers may be acquired and recorded to the test result retaining section 12. The records may then be analyzed by the analysis section 13 and by the test-result brain-state relationship information learning section 72 to visualize the burden on the healthcare personnel. The visualization may be implemented, for example, by the brain state outputting section 14 outputting relevant screens onto the brain state displaying section 24.

The results of the visualization are not limited to three-dimensional brain lesion and brain defect maps (brain maps). For example, the visualization results may include numerical data (scores) such as the volumes of the brain regions. Preferably, the method of displaying the brain maps may additionally include gray-scale indications reflecting the amounts of activities.

The information processing system 1 of this embodiment thus measures and outputs the severe part or the infarction (lesion) site and remaining functions of the brain using simplified tests and without necessarily measuring the brain.

All concepts and ideas of the present invention as represented by the above-described embodiment are obviously applicable to areas other than the brain, such as the tests on, and grasping of, the states of a living body.

As many apparently different embodiments of this invention may be made without departing from the spirit and scope thereof, it is to be understood that the invention is not limited to the specific embodiments thereof except as defined in the appended claims.

What is claimed is:

1. An information processing system comprising:
a storage section configured to store test-result brain-state relationship information associating results of activities of a plurality of examinees subjected to predetermined tests with brain states of the examinees;
an input section configured to accept a first test result as the result of the activity of a first examinee subjected to the predetermined tests;
a control section configured to estimate the brain state from the first test result on a basis of the test-result brain-state relationship information; and
an output section configured to output the estimated brain state.

2. The information processing system according to claim 1, wherein
the predetermined tests include tests on at least one of brain functions regarding motion, cognition, and attention,
given the first test result, the control section estimates a brain lesion site on the basis of the test-result brain-state relationship information, and
the output section outputs an image indicative of the brain lesion site.

3. The information processing system according to claim 2, wherein
the storage section includes:
a test result retaining section configured to retain the first test result; and
a test-result brain-state relationship information retaining section configured to retain the test-result brain-state relationship information, and
the control section estimates the brain state by inputting one or a plurality of parameters obtained from one or a plurality of test results into the test-result brain-state relationship information.

4. The information processing system according to claim 3, wherein
the control section includes a test-result brain-state relationship information learning section configured to learn relationship between a test result and a brain state by use of a database that includes relationships between the test results and the brain states, and
the test-result brain-state relationship information retaining section retails results of the learning by the test-result brain-state relationship information learning section.

5. The information processing system according to claim 2, wherein
the storage section retains a brain function database that associates brain sites with brain functions,
given the first test result, the control section estimates a brain site or a brain lesion site in which the activity of a brain function is reduced on a basis of the test-result brain-state relationship information and of the brain function database, and
the output section performs control to display, on a brain model, the estimated brain site or brain lesion site in which the activity of a brain function is reduced.

6. The information processing system according to claim 2, wherein
the storage section retains a brain function database that associates brain sites with brain functions and a rehabilitation database that associates a given brain function with a rehabilitation program for training the brain site related to the brain function,
given the brain lesion site, the control section references the brain function database to identify the brain function related to the brain lesion site, and
given the identified brain function, the output section references the rehabilitation database to output the rehabilitation program associated with the identified brain function.

7. The information processing system according to claim 1, wherein,
given the results of the predetermined tests performed at different times on the first examinee, the control section estimates the respective brain states at the different times on the basis of the test-result brain-state relationship information, and
the output section outputs the estimated respective brain states or changes over time in the brain state.

8. The information processing system according to claim 7, wherein
the output section outputs information on a rehabilitation program performed interposingly between the different times, together with the estimated respective brain states or the changes over time in the brain state.

9. The information processing system according to claim 1, wherein
the brain state output from the output section includes information on the brain lesion site estimated by the control section and on a reserve and remaining function of the brain.
